(19) United States
(12) Patent Application Publication
(10) Pub. No.: US 2007/0073230 A1
(43) Pub. Date: Mar. 29, 2007

(54) Drug infusions system with multiple medications

(75) Inventors: Keith E. Jasperson, Andover, MN (US); Thomas J. Valine, Spring Lake Park, MN (US); Frederic J. R. Wahlquist, Champlin, MN (US)

Correspondence Address: IPLM GROUP, P.A., POST OFFICE BOX 18455, MINNEAPOLIS, MN, 55418, US

(73) Assignee: Medtronic, Inc.

(21) Appl. No.: 11/600,631

(22) Filed: Nov. 16, 2006

Related U.S. Application Data
(63) Continuation of application No. 10/278,767, filed on Oct. 22, 2002.

(51) Int. Cl. A61M 37/00

(52) U.S. Cl. 604/131

(57) ABSTRACT

A drug infusion system is capable of delivering a fluid medication consisting of a plurality of drug to a patient under direction of a medical professional. The device is programmed to deliver a primary drug in a prescribed dose. The device determines the resultant dose of a secondary drug and displays such resultant dose to the medical professional. Dual sets of memory for storing operating parameters are alternatively active.

### RELATED APPLICATION

This application is a continuation of U.S. patent application Ser. No. 10/278,767, filed Oct. 22, 2002 and claims priority therefrom.

### FIELD OF THE INVENTION

This invention relates to drug infusion systems and, in particular, drug infusion systems that are programmable by a medical professional.

### BACKGROUND OF THE INVENTION

Drug infusion systems dispense fluid medication, containing a drug, to a patient. Some drug infusion systems are portable, allowing a patient to receive fluid medication while remaining mobile. In addition, some drug infusion systems are implantable to more effectively and less obtrusively dispense such fluid medication to a patient.

Implantable devices and techniques for treating a patient by drug infusion are well known in the prior art. For instance, U.S. Pat. No. 5,782,798, Rise, entitled Techniques For Treating Eating Disorders By Brain Stimulation and Drug Infusion; and U.S. Pat. No. 5,814,014, Elsberry et al, Techniques of Treating Neurodegnerative Disorders by Brain Infusion, each assigned to Medtronic, Inc., Minneapolis, Minn., disclose such devices and techniques and are hereby incorporated by reference.

Another example of a drug infusion device is shown in U.S. Pat. No. 3,527,220, Summers, entitled Implantable Drug Administrator, an implantable drug administrator having a refillable bladder which can be filled with a drug and a pump for selectively pumping the drug from the bladder into any desired area of the body. The administrator includes an indicator for indicating when the desired amount of the drug has been injected.

In U.S. Pat. No. 3,951,147, Tucker et al, entitled Implantable Infusate Pump, a rechargeable infusate pump for implantation in the human body can be refilled periodically by injection through an inlet septum under the skin. A conduit conducts fluid to an infusion site in the body. The pump outlet includes a special controller flow controller which is able to very accurately meter the infusate to the selected body site.

A problem with these implantable drug infusion devices is that there is no way to provide a simple external means to select the dosage amounts and intervals from a wide range of possible doses and intervals, and verify that a desired change had been made.

U.S. Pat. No. 4,146,029, Ellinwood, Self-Powered Implanted Programmable Medication System and Method, discloses a device and method for dispensing medication internally of the body utilizing an implanted system which includes medication storage and dispensing control circuitry having control components which may be modified by means external of the body being treated to control the manner of dispensing the medication within such body. In particular, extracorporeal control means may provide some measure to achieve selected medication programs corresponding to particular codes.

U.S. Pat. No. 4,692,147, Duggan, Drug Administration Device, assigned to Medtronic, Inc., Minneapolis, Minn., discloses an implantable drug administration device which can be non-invasively programmed to change both the dosage amount and the dosage interval. Verification of the received dosage and interval commands is achieved by means of an audio transducer which is attached to the device case.

The implantable drug administration device described in Duggan allows a medical professional to program to the delivery rate of a drug contained in the reservoir of the device over a specified interval.

Not infrequently, a medical professional prescribes more than one drug to be used in an implantable drug infusion device. More than one active ingredient present in the reservoir of the implantable infusion device increases programming difficulties substantially. Not only must the medical professional program the drug infusion device to perform a series programmed steps in order to deliver one drug to the patient, the medical professional must take into account the affect of creating or modifying a program for one of the drugs on the delivery of all other drugs also contained within the same reservoir of the drug infusion device. If the medical professional changes the delivery rate of the drug infusion device to increase the dose of one drug to be delivered to the patient in a period, that change will also increase the dose of all other drugs that are also contained in the same reservoir. With a complex dosing regimen and a plurality of active drugs, the danger for confusion and error is significant.

### BRIEF SUMMARY OF THE INVENTION

In one embodiment, the present invention provides a drug infusion system is capable of delivering a fluid medication to a patient under direction of a medical professional. The fluid medication consists of a plurality of drugs including a primary drug and a secondary drug. A drug delivery module is capable of delivering the fluid medication to the patient from a reservoir at a flow rate. A controller allows the medical professional to specify a dose of the primary drug per unit time for the patient. A flow rate is determined as a function of the dose of the primary drug and the concentration of the primary drug. A dose of the secondary drug per unit time for the patient is determined as a function of the flow rate and the concentration of the secondary drug. The controller communicates the dose for the secondary drug per unit time to the medical professional.

In a preferred embodiment, the concentration of each of the plurality of drugs is stored in the drug delivery module.

In a preferred embodiment, the controller communicates the dose for the secondary drug per unit time to the medical professional via a display.

In another embodiment, the present invention provides a drug infusion system capable of delivering a fluid medication to a patient under direction of a medical professional. The fluid medication consists of a plurality of drugs. A drug delivery module is capable of delivering the fluid medication to the patient from a reservoir. A controller allows the medical professional to specify a first parameter of delivery of one of the plurality of drugs. A first parameter of another of the plurality of drugs is determined as a function of the first parameter of delivery of the one of the plurality of drugs and a second parameter of another of the plurality of drugs. The controller communicates the first parameter of another of the plurality of drugs to the medical professional.

In a preferred embodiment, the drug infusion system further determines the flow rate as a function of the first parameter of the one of the plurality of drugs and a second parameter of the one of the plurality of drugs.

In a preferred embodiment, the first parameter of another of the plurality of drugs is determined as a function of the flow rate and the second parameter of another of the plurality of drugs.

In a preferred embodiment, the second parameter of each of the plurality of drugs is stored in the drug delivery module.

In a preferred embodiment, the drug delivery module is implantable.

In a preferred embodiment, the drug infusion system further determines an amount of the fluid medication contained in the reservoir.

In a preferred embodiment, the controller communicates the first parameter of another of the plurality of drugs to the medical professional via a display.

In another embodiment, the present invention provides a dosing tool, useable by a medical professional, for an implantable drug infusion system capable of delivering a fluid medication to a patient from a reservoir at a flow rate. The fluid medication consists of a plurality of drugs including a primary drug and a secondary drug. A controller allows the medical professional to specify a dose of the primary drug per unit time for the patient. The flow rate is determined as a function of the dose of the primary drug and the concentration of the primary drug. A dose of the secondary drug per unit time for the patient is determined as a function of the flow rate and the concentration of the secondary drug. The controller communicates the dose for the secondary drug per unit time to the medical professional.

In a preferred embodiment, the dosing tool further determines an amount of the fluid medication contained in the reservoir.

In a preferred embodiment, the controller communicates the dose for the secondary drug per unit time to the medical professional via a display.

In another embodiment, the present invention provides a method of communicating dosing information for an implantable drug infusion system to by medical professional when the drug infusion system is being programmed by the medical professional. The drug infusion system is capable of delivering a fluid medication to a patient from a reservoir at a flow rate. The fluid medication consists of a plurality of drugs including a primary drug and a secondary drug. The method allows the medical professional to specify a dose of the primary drug per unit time for the patient. The method determines the flow rate as a function of the dose of the primary drug and the concentration of the primary drug. The method determines a dose of the secondary drug per unit time for the patient as a function of the flow rate and the concentration of the secondary drug. The method communicates the dose for the secondary drug per unit time to the medical professional.

In a preferred embodiment, the method further determines an amount of the fluid medication contained in the reservoir.

In a preferred embodiment, the communication step is accomplished via a display.

In another embodiment, the present invention provides a drug infusion system capable of delivering a fluid medication to a patient under direction of a medical professional. An implantable drug delivery module, having operating parameters, is capable of delivering the fluid medication to the patient. A memory, contained in the implantable drug delivery module, stores a plurality of sets of the operating parameters, one of the plurality of sets of the operating parameters being active. A controller allows the medical professional to specify the operating parameters by modifying the parameters stored in one of the plurality of sets of the operating parameters which is not active. The controller also allows the medical professional to alter which of the plurality of sets of operating parameters is active.

In another embodiment, the present invention provides a method of controlling a drug infusion system capable of delivering a fluid medication to a patient under direction of a medical professional. An implantable drug delivery module is capable of delivering the fluid medication to the patient, the implantable drug delivery module having operating parameters. Memory, contained in the implantable drug delivery module, stores a plurality of sets of the operating parameters, one of the plurality of sets of the operating parameters being active. The method stores a set of operating parameters in the memory in one of the plurality of sets which is not active. The method determines that the set of operating parameters is valid. The method switches which of the plurality of sets which is active to the one of the plurality of sets of operating parameters in which the set of operating parameters were stored in the storing step.

### BRIEF DESCRIPTION OF THE DRAWINGS

FIG. 1 is a schematic view of a patient with a drug infusion device implanted within the patient's body.

FIG. 2 is a block diagram of a drug infusion device of the present invention;

FIG. 3 is a block diagram illustrating the random access memory and register layout of a portion of drug infusion device of the present invention;

FIG. 4 is an illustration of a drug entry display provided to a programmer of the drug infusion system of the present invention;

FIG. 5 is another illustration of a drug entry display provided to a programmer of the drug infusion system of the present invention;

FIG. 6 is an illustration of a drug delivery display provided to a programmer of the drug infusion system of the present invention showing simple continuous mode programming;

FIG. 7 is another illustration of a drug delivery display provided to a programmer of the drug infusion system of the present invention showing simple continuous mode programming; and

FIG. 8 is an illustration of a drug delivery display provided to a programmer of the drug infusion system of the present invention showing flex mode programming.

### DETAILED DESCRIPTION OF THE INVENTION

The entire contents of U.S. application Ser. No. 10/278,767, filed Oct. 22, 2006, is hereby incorporated by reference.

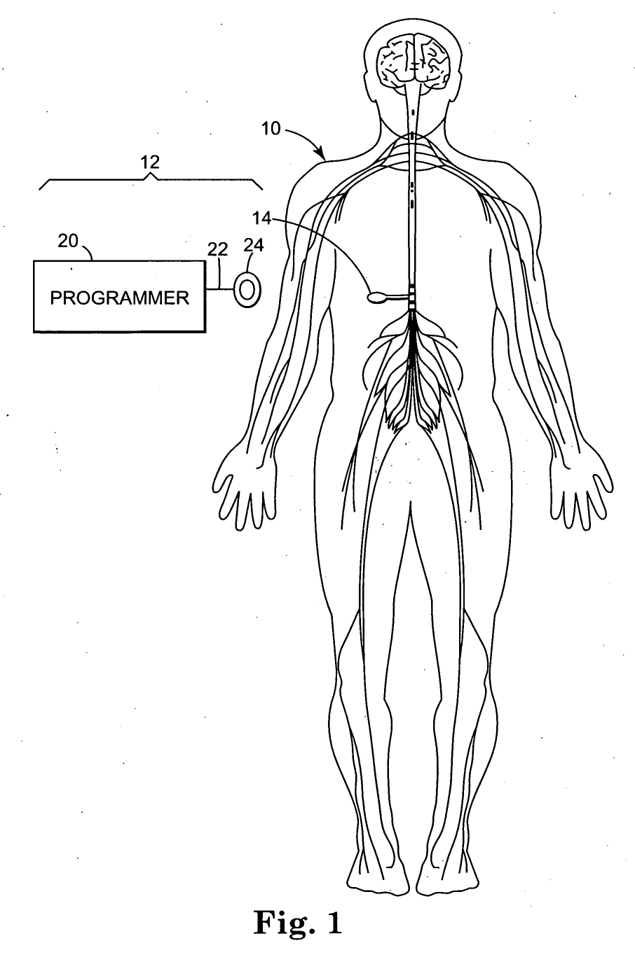

FIG. 1 is a schematic view of a drug infusion system 12 of the present invention. Implantable drug infusion device 14 is shown implanted within the body of patient 10. Drug infusion device 14 is programmable through a telemetry link from programmer 20, which is coupled via a conductor 22 to a radio frequency antenna 24. Methods of communicating, using radio frequency telemetry, with implanted treatment devices in order to program such implanted drug infusion devices, are well known in the art.

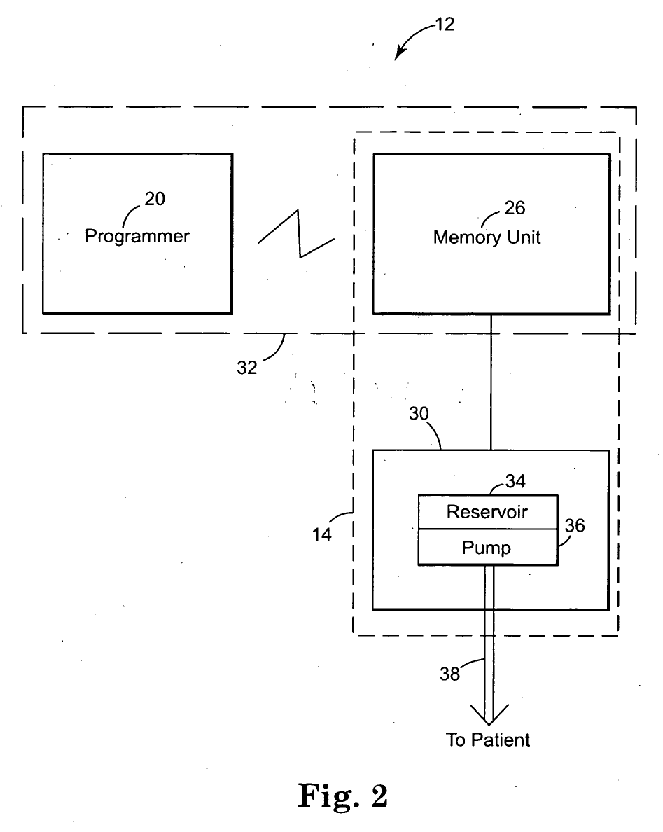

FIG. 2 is a block diagram of the drug infusion system 12 having an implantable drug infusion device 14. Drug infusion device 14 consists of an internal memory unit 26 containing memory and registers and circuitry which provides internal drug delivery instructions to drug delivery module 30. External programmer 20 acts as an input-output device for drug infusion system and also provides computational support for memory unit 26. Memory unit 26 and programmer 20, operating together, function as a controller 32 controlling drug delivery module 30 in the delivery of fluid medication to patient 10. Drug delivery module 30 has a reservoir 34 for holding the fluid medication to be infused and pump 36 coupled to patient 10 through catheter tubing 38. Such drug delivery modules 30 are well known in the art.

Memory unit 26 receives programming information, via telemetry, from programmer 20 through conventional means. Programming information, once stored in memory unit 26, provides the dosing regimen to be performed by drug delivery module 30.

Memory unit 26 stores information concerning aspects of the operation of drug infusion device 14. Memory unit 26 may, for example, store information about patient 10 including name, address and contact information. In addition, memory unit 26 may store information about the drug delivery module 30 including pump 36 and catheter 38. Among the settings that may be stored are the model number and serial number of pump 36, the volume of reservoir 34, battery condition and information about catheter 38, including the length of all sections of catheter 38. Information about the calibration of pump 36 may also be stored. During the pumping operation, controller 32 (preferably through programmer 20) also calculates, or otherwise determines, the volume of fluid medication remaining in reservoir 34.

In order to perform its function, memory unit 26 also stores information about each drug contained in reservoir 34, including the drug name, or other identifier, and the concentration of the drug in the overall volume of fluid medication contained in reservoir 34. Typically, this data is entered at the time that drug infusion device 14 is loaded with fluid medication.

Throughout this description, while it is contemplated that any or all of the calculations performed by controller 32 could be performed in either programmer 20 or memory unit 26, it is recognized that drug delivery device 14, being an implantable device, will have a limited amount of processing power and energy source. Therefore, it is preferred that the calculations referred to as being performed by controller 32 (encompassing both programmer 20 and memory unit 26) actually be performed by programmer 20, in order to control the precious resources of implantable drug infusion device 14. At the same time, it is preferred that information concerning implantable drug infusion device 14, patient 10, all drug contained in reservoir 34 and the drug regimens implemented, all be stored in memory unit 26 so as to be available no matter which of a plurality of programmers 20 may be operationally coupled with implantable drug infusion device 14 to form drug infusion system 12.

Alternatively, the amount of drug, e.g., in micrograms, can be entered into controller 34 when drug infusion device is loaded with fluid medication. That information, along with the also known volume of fluid medication, e.g., 12 milliliters, allows controller 34 to calculate the concentration of the drug. In the example given above, if the volume of fluid medication is 12 milliliters and the amount of drug is 20 micrograms, then the concentration the drug 1.666 micrograms per milliliter (20 micrograms divided by 12 milliliters).

Drug infusion device 14 may also contain (and be programmed for) more than one drug. Typically, a multiple drug prescription, the drug cocktail containing multiple drugs, or an active drug and a neutral agent such as saline, is premixed and then injected into the implanted drug infusion device. The concentration of each of the drugs contained in the drug cocktail is known, or the amount of each of the drugs contained in the drug cocktail is known, or a combination of the above. The requisite information for each of the drugs contained in the drug cocktail are entered into memory unit 26 in the same way as described above with respect to one drug.

In a preferred embodiment, the patient information, pump information and the drug information is all stored in memory unit 26 located in an implanted drug infusion device 14. While all of this information readily available from within implanted drug infusion device 14, a medical professional may use any applicable programmer 20, at any time and any location, to read information from implanted drug infusion device 14 and to program drug infusion device 14.

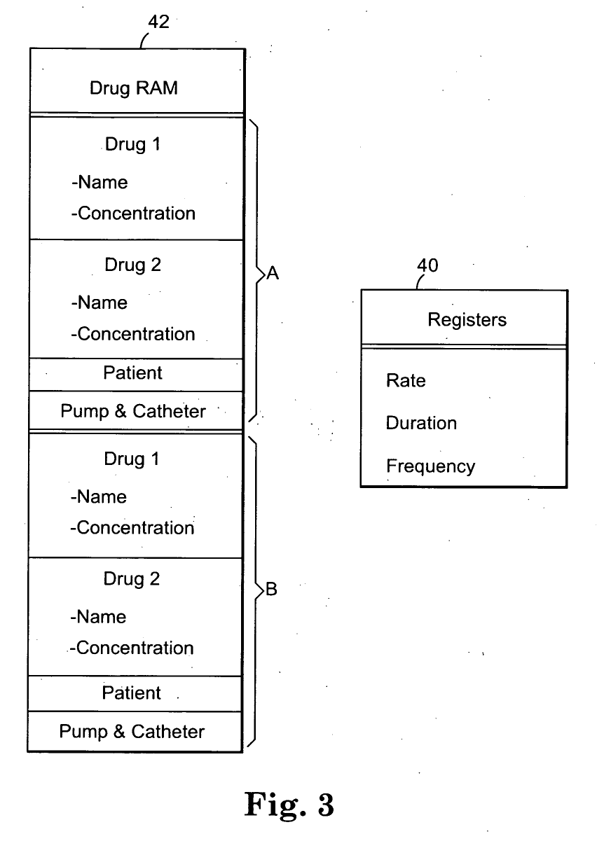

FIG. 3 illustrates a preferred manner of storing such information within memory unit 26 of drug infusion device 14. Registers 40 contain information concerning programmed drug regimens, including the number of steps, their frequency (if repetitive), duration and pumping rate. Such information is generic to all drugs contained in reservoir 34 irrespective of the nature of the drug or drugs contained in reservoir 34 or the number of drugs contained in reservoir 34. In general, the information contained in registers 40 represents the information which applies whichever drug is contained in reservoir 34 or applies equally to all drugs in reservoir 34. For example, the delivery rate represents the programmed rate at which pump 36 delivers fluid medication to patient 10. Since all of the drugs present in reservoir 34 of drug infusion device 14 are delivered to patient from common reservoir 34 at whatever rate pump 36 is programmed, all drugs are delivered to patient 10 at precisely the same rate. Hence, information about delivery rate, pumping rate, may be stored in registers 40 and can be common to all drugs contained in drug infusion device 14.

Drug RAM (random access memory) 42 holds information about each of the drugs which are contained in reservoir 34 of drug infusion device 14. As noted above, such drug information includes the name, or other identifier, of each individual drug as well as the concentration of the drug in total fluid volume of fluid medication and/or the amount of such drug contained in reservoir 34.

In a preferred embodiment, drug RAM 42 is separated into two at least two parts, labeled part A and part B. Parts A and B of drug RAM 42 are identical and each contains exactly the same type of information, although, of course, the data contained in each individual memory location or locations may differ. Thus, parts A and B of drug RAM 42 are completely redundant. However, only either part A or part B of drug RAM 42 is active at any one time.

The information in part A when active, for example, of drug RAM 42, in conjunction with the information contained in registers 40, specifies the operating parameters and controls the operation of drug delivery module 30 and, therefore, delivers the proper amount of fluid medication to patient 10 at the proper time. In this case, the operating parameters in part B of drug RAM 42 are inactive and do not control the operation of drug infusion device 14. Conversely, when the operating parameters stored in part B of drug RAM 42 are active, the information contained therein, along with the information contained in registers 40, controls the operation of drug infusion device 14 and the operating parameters stored in part A of drug RAM 42 are inactive and do not control any aspect of the operation of drug infusion device 14. Thus, parts A and B are completely redundant and alternatively control the operation of drug infusion device 14.

When new information about the drugs contained in reservoir 34 of drug infusion device 14, patient information or information about pump 36 and/or catheter 38 is written to drug RAM 42, it is written to the part of drug RAM 42 which is not, at that time, active.

Mainly due to the amount of information which may need to be written t1 the inactive portion of drug RAM 42, e.g., information about multiple drugs including their name and concentration, the writing of such information may need to be performed in separate write steps or, in other words, in separate packets of information. Because the information is not written in a simple step, or in a single packet, there exists the possibility that the writing process may be interrupted. This could occur, for example, if communication with implantable drug infusion device 14 was lost due to movement of patient 10 or of programmer 20 or could occur if battery power were lost to programmer 20.

Since the information about drugs, patient and pump 36 and/or catheter 38 are written to the portion of drug RAM 42 which is not currently active, the inactive portion of drug RAM 42 serves as a shadow RAM to hold such information until the entire writing process can be finished. Registers 40 can then be updated to transfer control of drug infusion device 12 from the previously active portion of drug RAM 42 to the newly written and previously inactive portion of drug RAM 42.

Information about the rate, duration and frequency of each step of drug delivery programmed into drug infusion device 12 is contained in registers 40. The rate, duration and frequency information contained in registers 40, along with the information contained in drug RAM 42 and information contained in registers 40 on which portion of drug RAM 42 is active, determine the operation of drug infusion device 12. In a preferred embodiment, new information written into registers 40 concerning rate, duration, frequency and which portion of drug RAM 42 is active, is written as a single step. In other words, this information is written as a single packet of data. Thus, there is no possibility that some of the information will be written and the writing process will be interrupted. Thus, new information, if needed, is written into the inactive portion of drug RAM 42 first and then new information, if needed, is written into registers 40 and control is transferred from the previously active portion of drug RAM 42 to the previously inactive portion of drug RAM 42. This stepped process enables information from a plurality of writing steps to be transferred to the memory which controls drug infusion device 12 without the danger that an interruption in writing process will result in only a portion of the intended new information controlling drug infusion device 12.

It is also to be recognized and understood that while registers 40 and drug RAM 42 of memory unit 26 have been described, in a preferred embodiment, as having two parts, namely parts A and B, that the same principles apply and registers 40 and drug RAM 42, or either of them, may be separated into more than two parts with equally advantageous operating results.

The operating parameters stored in each part (part A and part B) of drug RAM 42 of memory unit 26 may be again divided into separate areas. Each part of drug RAM 42 contains information relating to each of the drugs contained in reservoir 34. For example, if two drugs are contained in reservoir 34 (and part A is active), then part A of drug RAM 42 will be divided into sections. There is a section devoted to information about drug one and a section devoted to information about drug two. And, of course, drug RAM 42 may be separated into any multiple of parts, at least one for each of the number of drugs which are contained, or which may be contained, in reservoir 34.

If more than one drug is prescribed for drug infusion system 12, the proper amount of each drug will typically be pre-mixed before insertion into reservoir 34 of implantable drug infusion device 14. Each drug in the mixture will have a concentration, i.e., an amount of each drug compared to the overall volume of fluid medication contained, to be contained, in reservoir 34. At or near the time that the drug mixture containing the multiple drugs is inserted into reservoir 34, usually through a syringe for an implantable drug infusion device 14, data concerning all of the drugs contained in fluid medication is entered into memory unit 26.

Generally, one of the drugs contained in fluid medication is the primary drug. The primary drug is main drug around which the prescription drug mix, or drug cocktail, is based. It is the primary drug on which the basic programming decisions for drug infusion system 12 are based.

While one drug may be the primary drug contained in the fluid medication, the medical professional must not overlook the effects of other drugs contained in the fluid medication. If the prescription for the amount of the primary drug in increased, typically by increasing the delivery rate of pump 36, the amount of all other drugs, which are contained in fluid medication, delivered to patient 10 will also be increased. Thus, the medical professional must take into account all of the drugs contained in fluid medication. If the dose for the primary drug is changed, then the dose for all of the drugs will be changed. If the medical professional does not keep track of the affect of modifying the delivery rate on all of the drugs contained in the fluid medication, the patient 10 may receive more or less of the other drugs contained in the fluid medication.

Programmer 20 portion of controller 32 provides an interface between the medical professional and the potentially implanted drug infusion device 14. In particular, programmer 20 provides a medium for data entry into memory 26 of drug infusion device 14 and provides a display for communication of information contained in memory 26 to the medical professional. As noted above, programmer 20 also, preferably, provides computational power to perform the calculations associated with drug infusion system 12.

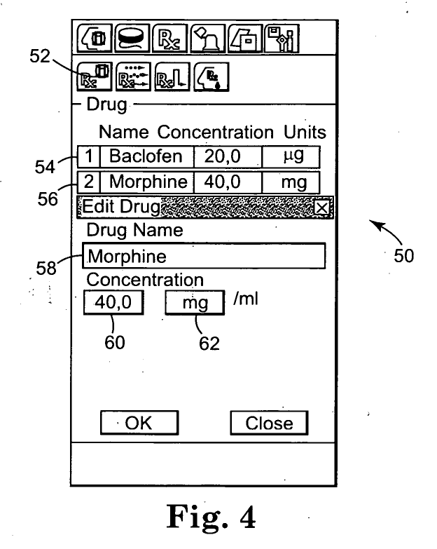

FIG. 4 illustrates an “input-output” display 50 on programmer 20 with the drug tab 52 selected. Display 50, associated with drug tab 52, provides a mechanism for the medical professional to input information about the drugs contained in the fluid medication to controller 32. Display 50 also provides a mechanism for the medical professional to learn with what drugs controller 32 has been programmed.

Drug entry display has an entry (54, 56) for each of the multiple drugs contained fluid medication. Entry number 1 (54) contains information on the primary drug including the name of the drug and the concentration of the drug. In the embodiment illustrated in FIG. 4, entry 54 contains Baclofen. Entry 54 also contains information about the concentration of the primary drug Baclofen, here listed as 20.0 micrograms. This concentration means the fluid medication has 20.0 micrograms of Baclofen per milliliter of fluid medication. Entry 56 illustrates the entry of information about drug 2, a secondary drug in fluid medication. In the embodiment illustrated in FIG. 4, entry 56 contains morphine. The information about secondary drug morphine is expanded from its read-only status at entry 56 to data entry dialog boxes 58, 60 & 62. Dialog box 58 appears to facilitate entry of the actual name of morphine as a secondary drug. This is the spot that the medical professional enters this information. Similarly, dialog box 60 facilitates entry of amount of the concentration of morphine and dialog box 62 facilitates entry of the units associated with the amount of the concentration of morphine. In the example illustrated in FIG. 4, secondary drug morphine has a concentration of 40.0 milligrams per milliliter of fluid medication. Once the medical professional is assured that the information contained in dialog boxes 58, 60 & 62 are correct, the “OK” box can be clicked the entry 56 will be updated with the proper information from dialog boxes 58, 60 & 62.

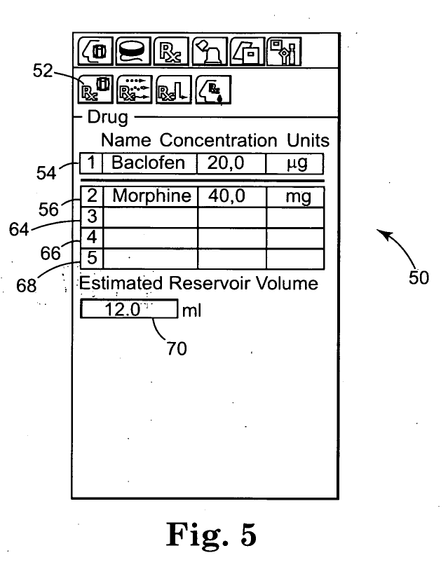

FIG. 5 illustrates the appearance of drug entry display 50 with dialog box 58, 60 & 62 closed. Entries 54 and 56 appear as they did in FIG. 4. In addition, drug entry display 50 illustrated in FIG. 5 contains spaces for entries 64, 66 & 68. Since, only two drugs are contained in fluid medication in the example illustrated in FIG. 5, entries 64, 66 & 68 are empty. If, however, fluid medication contained more than two drugs, information about the additional drugs would be contained in entries 64 (if there were a total of three drugs), 64 and 66 (if there were a total of four drugs) and entries 64, 66 and 68 (if there were total of five drugs). Of course, additional drugs could be accommodated with additional entries.

In addition to information about each individual drug contained in fluid medication, display 50 also contains information (entry 70) about the estimated volume of fluid medication contained in reservoir 34. The initial value entered into entry 70 would be the total amount of fluid medication which is, or is to be, inserted into reservoir 34 of drug infusion device 14. After initial entry of the value contained in entry 70, drug infusion device 14 calculates the amount fluid medication which has been delivered by drug infusion device 14 and subtracts that amount from the initial value entered into entry 70. Drug infusion device, via controller 34, then causes the updated amount of fluid medication remaining in reservoir 34 (as calculated above) to be displayed in entry 70.

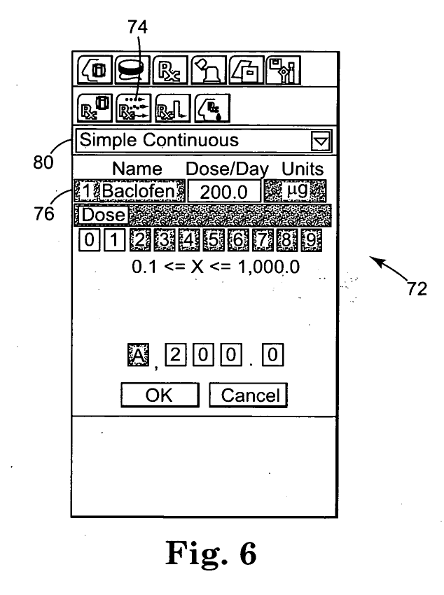

FIG. 6 illustrates drug delivery display 72 as selected by drug delivery tab 74. Drug delivery display 72 has been selected, via delivery mode drop-down box 80, to be in “simple continuous” mode, meaning that pump 36 is programmed to delivery fluid medication at a constant rate. Drug delivery display 72 has entry 76 which is indicative of the prescribed dose of the primary drug (entered in drug entry display screen 50). In this part of the example, the primary drug is identified in entry 76 as Baclofen. The data entry in the lower portion of drug delivery display 72 is open and illustrates the entering of a daily dose of Baclofen (drug 1, the primary drug) of 200 micrograms.

The daily dose of the primary drug entered in drug delivery display 72 converted by controller 32, preferably programmer 20, into a drug delivery rate, i.e., the rate at which pump 36 delivers the fluid medication to patient 10, and is transferred to memory unit 26. Controller 32 calculates the rate at which pump 36 delivers fluid medication to patient 10 by converting, if necessary, the daily dose the primary drug into a dose of the primary drug in a smaller unit of time. This amount of dose is then divided by the concentration of the primary drug in the fluid medication contained in reservoir 34 resulting in an amount of the fluid medication to be delivered to patient 10 over that unit of time. Pump 36 is then set to deliver the fluid medication at that rate.

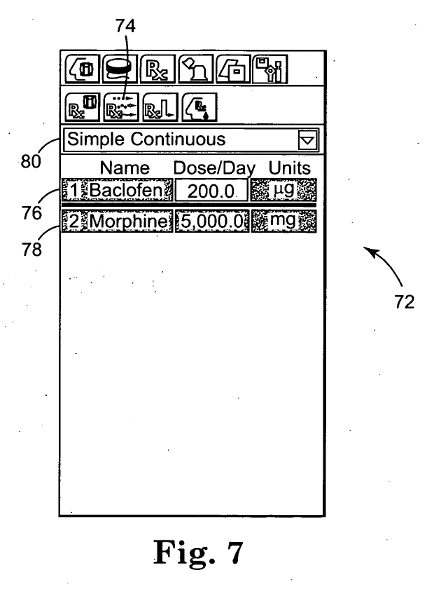

FIG. 7 illustrates drug delivery display 72 also as selected by drug delivery tab 74 and also in simple continuous mode as selected by delivery mode drop-down box 80. Drug delivery display 72 illustrated in FIG. 7 differs from drug delivery display 72 illustrated in FIG. 6 in that the data entry in the lower portion of drug delivery display 72 is now closed, the data for the drug delivery rate for primary drug Baclofen having been entered in entry 76. Closing of the data entry portion allows drug delivery display 72 not only entry 76 for primary drug Baclofen but also allows display entry 78 for secondary drug identified as morphine in this portion of the example.

Drug dose entry 76 displays the secondary drug name, morphine, and the daily dose for the secondary drug, here 5,000 milligrams. In contrast to drug dose entry 76 for primary drug Baclofen, drug dose delivery entry 78 for secondary drug morphine is not directly entered by the medical professional. Since the medical professional has already set the rate at which pump 36 delivers fluid medication to patient 10 via drug dose delivery rate 76, all other drugs in fluid medication will be delivered at that same rate. Hence, drug dose entry 78 is instead an informational display of a calculated daily dose for secondary drug morphine for the medical professional.

The daily dose of secondary drug, morphine, displayed here in entry 78 is calculated by controller 32. The rate at which pump 36 is set to deliver the fluid medication to patient 10 is known by the calculation resulting from the dosing programmed for the primary drug, Baclofen. The rate at which pump 36 is set to deliver fluid medication to patient 10 per unit time is multiplied by the concentration -of the secondary drug in the fluid medication contained in reservoir 34. This results in the dose of the secondary drug set to be delivered to patient 10 per unit of time. The dose is then converted into a daily dose simply by adjusting the time scale, if necessary. The resulting daily dose is then displayed in entry 78 of drug dose display 72.

While FIGS. 6 and 7 have been illustrated with two drugs, a primary drug and a secondary drug, it is to be recognized and understood that more secondary drugs could also be mixed in fluid medication and, hence, also be added as additional entries in drug delivery display 72. A drug contained in fluid medication will have an entry in both drug entry display 50 and drug delivery display 72.

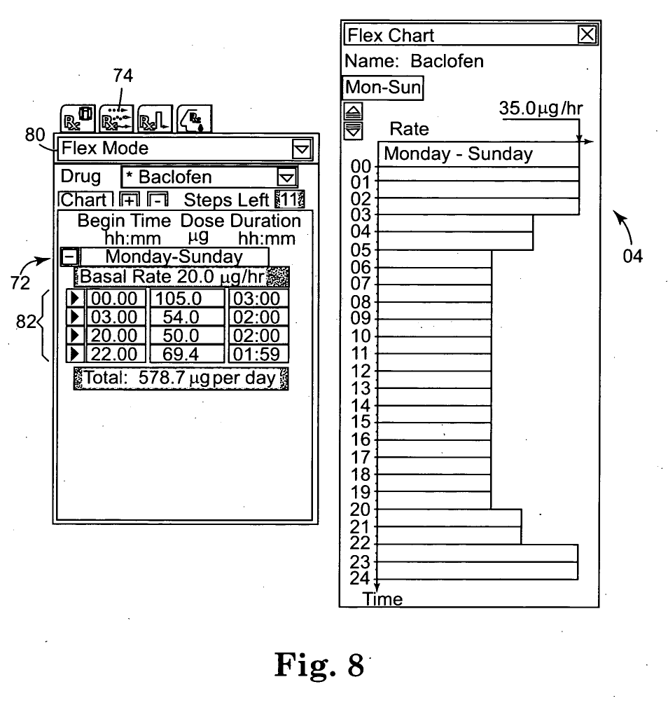

FIG. 8 illustrates drug delivery display 72, as selected by drug delivery tab 74, this time illustrating the programming of the delivery of primary drug Baclofen in “flex mode” as selected by drop-down box 80. In flexible mode, the delivery rate for the primary drug can be varied based on multiple time periods, such as particular hours, in a longer time period, such as a day. The variation in drug delivery rate can be entered directly by particular interval step as shown at 82 or can be displayed and/or entered as illustrated in the alternative graphical representation 84.

Again, once the delivery mode and rate for the primary drug has been entered, controller 32 of drug infusion device 14 calculates and displays, via drug delivery display 72, the daily dose and drug delivery particulars, if in flexible mode, of a secondary drug or drugs.

By displaying the daily dosage or particular delivery rates for a secondary drug or drugs directly in the display of programmer 20, the medical professional is kept apprised of the affect of dosing decisions based upon the primary drug on dosing for a secondary drug or drugs. The makes programming drug infusion device easier, more straightforward and helps eliminate errors and dosing miscalculations.

Thus, embodiments of the drug infusion device with multiple medications are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A drug infusion system capable of delivering a fluid medication to a patient under direction of a medical professional, said fluid medication consisting of a plurality of drugs including a primary drug and a secondary drug, each of said plurality of drugs having a concentration, said drug infusion device comprising:
a drug delivery module capable of delivering said fluid medication to said patient from a reservoir at a flow rate;
a controller allowing said medical professional to specify a dose of said primary drug per unit time for said patient;
means for determining said flow rate as a function of said dose of said primary drug and said concentration of said primary drug;
means for determining a dose of said secondary drug per unit time for said patient as a function of said flow rate and said concentration of said secondary drug;
said controller communicating said dose for said secondary drug per unit time to said medical professional.

2. A drug infusion system as in claim 1 wherein said concentration of each of said plurality of drugs is stored in said drug delivery module.

3. A drug infusion system as in claim 1 wherein said drug delivery module is implantable.

4. A drug infusion system as in claim 1 which further comprises means for determining an amount of said fluid medication contained in said reservoir.

5. A drug infusion system as in claim 1 wherein said concentration of each of said plurality of drugs is stored in said drug delivery module.

6. A drug infusion system as in claim 1 wherein said controller communicates said dose for said secondary drug per unit time to said medical professional via a display.

7. A drug infusion system capable of delivering a fluid medication to a patient under direction of a medical professional, said fluid medication consisting of a plurality of drugs, said drug infusion system comprising:
a drug delivery module capable of delivering said fluid medication to said patient from a reservoir;
a controller allowing said medical professional to specify a first parameter of delivery of one of said plurality of drugs; and
means for determining a first parameter of another of said plurality of drugs as a function of said first parameter of delivery of said one of said plurality of drugs and a second parameter of said another of said plurality of drugs;
said controller communicating said first parameter of said another of said plurality of drugs to said medical professional.

8. A drug infusion system as in claim 7 wherein said drug infusion system further comprises means for determining said flow rate as a function of said first parameter of said one of said plurality of drugs and a second parameter of said one of said plurality of drugs.

9. A drug infusion system as in claim 8 wherein said first parameter of said another of said plurality of drugs is determined as a function of said flow rate and said second parameter of said another of said plurality of drugs.

10. A drug infusion system as in claim 7 wherein said second parameter of each of said plurality of drugs is stored in said drug delivery module.

11. A drug infusion system as in claim 7 wherein said drug delivery module is implantable.

12. A drug infusion system as in claim 7 which further comprises means for determining an amount of said fluid medication contained in said reservoir.

13. A drug infusion system as in claim 7 wherein said controller communicates said first parameter of said another of said plurality of drugs to said medical professional via a display.

14. A dosing tool, useable by a medical professional, for an implantable drug infusion system capable of delivering a fluid medication to a patient from a reservoir at a flow rate, said fluid medication consisting of a plurality of drugs including a primary drug and a secondary drug, each of said plurality of drugs having a concentration, said dosing tool comprising:
a controller allowing said medical professional to specify a dose of said primary drug per unit time for said patient;
means for determining said flow rate as a function of said dose of said primary drug and said concentration of said primary drug; and
means for determining a dose of said secondary drug per unit time for said patient as a function of said flow rate and said concentration of said secondary drug;
said controller communicating said dose for said secondary drug per unit time to said medical professional.

15. A dosing tool as in claim 14 which further comprises means for determining an amount of said fluid medication contained in said reservoir.

16. A dosing tool as in claim 14 wherein said controller communicates said dose for said secondary drug per unit time to said medical professional via a display.

17. A method of communicating dosing information for an implantable drug infusion system to by medical professional when said drug infusion system is being programmed by said medical professional, said drug infusion system being capable of delivering a fluid medication to a patient from a reservoir at a flow rate, said fluid medication consisting of a plurality of drugs including a primary drug and a secondary drug, each of said plurality of drugs having a concentration, said method comprising the steps of:
allowing said medical professional to specify a dose of said primary drug per unit time for said patient;
determining said flow rate as a function of said dose of said primary drug and said concentration of said primary drug;
determining a dose of said secondary drug per unit time for said patient as a function of said flow rate and said concentration of said secondary drug; and
communicating said dose for said secondary drug per unit time to said medical professional.

18. A method as in claim 17 which further comprises the step of determining an amount of said fluid medication contained in said reservoir.

19. A method as in claim 17 wherein said communication step is accomplished via a display.

20. A drug infusion system capable of delivering a fluid medication to a patient under direction of a medical professional, comprising:
an implantable drug delivery module capable of delivering said fluid medication to said patient, said implantable drug delivery module having operating parameters;
memory, contained in said implantable drug delivery module, for storing a plurality of sets of said operating parameters, one of said plurality of sets of said operating parameters being active;
a controller allowing said medical professional to specify said operating parameters by modifying said parameters stored in one of said plurality of sets of said operating parameters which is not active;
said controller also allowing said medical professional to alter which of said plurality of sets of operating parameters is active.

21. A method of controlling a drug infusion system capable of delivering a fluid medication to a patient under direction of a medical professional, said drug infusion system having:
an implantable drug delivery module capable of delivering said fluid medication to said patient, said implantable drug delivery module having operating parameters; and
memory, contained in said implantable drug delivery module, for storing a plurality of sets of said operating parameters, one of said plurality of sets of said operating parameters being active;
comprising the steps of:
storing a set of operating parameters in said memory in one of said plurality of sets which is not active;
determining that said set of operating parameters is valid; and
switching which of said plurality of sets which is active to said one of said plurality of sets of operating parameters in which said set of operating parameters were stored in said storing step.
